(19) United States
(12) Patent Application Publication
(10) Pub. No.: US 2022/0211332 A1
(43) Pub. Date: Jul. 7, 2022

(54) MEDICAL DEVICE SYSTEM FOR MONITORING PATIENT HEALTH

(71) Applicant: Medtronic, Inc., Minneapolis, MN (US)

(72) Inventors: Wade M. Demmer, Coon Rapids, MN (US); Vinod Sharma, Maple Grove, MN (US); Shantanu Sarkar, Roseville, MN (US); James R Peichel, Minneapolis, MN (US)

(21) Appl. No.: 17/647,314

(22) Filed: Jan. 6, 2022

Related U.S. Application Data
(60) Provisional application No. 63/134,767, filed on Jan. 7, 2021.

(51) Int. Cl. A61B 5/00; A61B 5/11; A61B 7/00; A61B 5/361; A61B 5/0538; A61B 5/0205; A61B 5/024; G16H 40/67; G16H 10/20; G16H 50/30

(52) U.S. Cl. CPC A61B 5/7275; A61B 5/0002; A61B 5/742; A61B 5/1112; A61B 7/00; A61B 5/0077; G16H 50/30; A61B 5/0538; A61B 5/02055; A61B 5/02405; A61B 5/1118; G16H 40/67; G16H 10/20; A61B 5/361

(57) ABSTRACT

A method of monitoring a patient using a system includes a medical device, a peripheral device configured to wirelessly communicate with the medical device, and processing circuitry. The method includes, by the processing circuitry, receiving sensor data collected by the medical device and evaluating the sensor data. The method further includes, based on the evaluation of the sensor data, outputting for display via the peripheral device at least one question relating to the sensor data collected by the medical device for a patient to answer. The method further includes receiving at least one answer via the peripheral device and determining, based on a combination of the sensor data and the at least one answer, a risk-level of the patient's health associated with at least one condition such as at least one of infection, stroke, sepsis, chronic obstructive pulmonary disease, cardiac arrhythmia, or myocardial infarction.

This application claims the benefit of U.S. Provisional Application Ser. No. 63/134,767, filed Jan. 7, 2021, the entire content of which is incorporated herein by reference.

### FIELD

This disclosure relates to medical devices and computing devices for monitoring a patient's health.

### BACKGROUND

Medical devices may be used to monitor and/or treat a variety of medical conditions. Example medical devices include implantable medical devices (IMDs), such as cardiac or cardiovascular implantable electronic devices (CIED), and external medical devices. An IMD may include a device implanted in a patient at a surgically or procedurally prepared implantation site.

An IMD may sense physiological activity of the patient via electrodes and/or at least one sensor included within or coupled to the IMD. The IMD may be configured to deliver therapy to the patient, such as electrical stimulation therapy via electrodes, where the IMD may be configured to stimulate the heart, nerves, muscles, brain tissue, etc. The IMD may, in some instances, include a battery powered component including electronics and a battery within a housing. In such instances, the battery powered component of the IMD may be implanted, such as at a surgically or procedurally prepared implantation site. In addition, associated devices, such as elongated medical electrical leads or drug delivery catheters, can extend from the IMD to other subcutaneous implantation sites or in some instances, deeper into the body, such as to organs or various other implantation sites. In some examples, the IMD need not be coupled to leads and may include a battery powered component implanted subcutaneously or deeper into the body.

Inevitably, patients who have IMDs have medical issues that require a visit with a healthcare professional (HCP), such as a physician. The HCP visit scheduling may take a few days to a few weeks after the patient notices the first symptoms. The HCP typically performs a wellness check and possibly initiates a device interrogation session to determine performance metrics of the IMD. In some cases, this visit uncovers potential health issues or device-related complications that potentially warrant clinical intervention.

### SUMMARY

While visits with the healthcare professional (HCP) tend to be relatively short, the visits can still constitute a burden on the patient's life, the physician's clinic, and on the healthcare system overall. Aspects of this disclosure are directed to techniques for monitoring a patient using a medical device, a peripheral device configured to wireles sly communicate with the medical device, and processing circuitry. The medical device, and in some cases multiple medical devices, may collect sensor data of the patient, which may be evaluated by the processing circuitry. Based on the evaluation of the sensor data, the peripheral device may pose at least one question relating to the sensor data for the patient, or another user such as a caregiver of the patient, to answer. Based on a combination of the sensor data and the at least one answer, the processing circuitry may determine a risk-level of the patient's health associated with at least one condition, such as infection, stroke, sepsis, heart failure, chronic obstructive pulmonary disease (COPD), heart failure decompensation, cardiac arrhythmia, or myocardial infarction. The use of combined data streams from medical device sensors, peripheral device sensors, and/or the answers may advantageously provide a more holistic and complete picture of patient status. Triggering the presentation of the questions based on an analysis of sensor data may allow the questions to be advantageously posed at a relevant time.

In some examples, a method for monitoring a patient uses a system including a medical device, a peripheral device configured to wirelessly communicate with the medical device, and processing circuitry. The method includes, by the processing circuitry, receiving sensor data collected by the medical device and evaluating the sensor data. The method further includes, based on the evaluation of the sensor data, outputting for display via the peripheral device at least one question relating to the sensor data collected by the medical device for a patient to answer. The method further includes receiving at least one answer via the peripheral device and determining, based on a combination of the sensor data and the at least one answer, a risk-level of the patient's health associated with at least one of infection, stroke, sepsis, heart failure, COPD, heart failure decompensation, cardiac arrhythmia, or myocardial infarction.

In some examples, the method may further include confirming, by the processing circuitry, accuracy of the sensor data using the patient's at least one answer to the at least one question. Additionally or alternatively, the method may further include monitoring, by the processing circuitry, the patient, where at least one of a period or a frequency of monitoring are based on the risk level of the patient's health. Additionally or alternatively, the method may further include outputting for display, by the processing circuitry and based on the risk-level of the patient's health, at least one of the at least one question about the patient's health, an indication of the risk level of the patient's health, a notification to maintain status quo, a recommendation to increase monitoring, or an alert to contact a clinician.

In some examples, a system for monitoring a patient may include a medical device configured to collect sensor data related to at least one physiological parameters of the patient, a peripheral device configured to wirelessly communicate with the medical device, and processing circuitry. The processing circuitry is configured to receive sensor data collected by the medical device, evaluate the sensor data, based on the evaluation of the sensor data, output for display via the peripheral device at least one question relating to the sensor data collected by the medical device for a patient to answer. The processing circuitry is further configured to receive at least one answer via the peripheral device and determine, based on a combination of the sensor data and the at least one answer, a risk-level of the patient's health associated with at least one of infection, stroke, sepsis, heart failure, COPD, heart failure decompensation, cardiac arrhythmia, or myocardial infarction.

In some examples, a computer-readable medium may include instructions that, when executed, cause processing circuitry to receive sensor data collected by the medical device, evaluate the sensor data, based on the evaluation of the sensor data, output for display via the peripheral device at least one question relating to the sensor data collected by the medical device for a patient to answer. The instructions further cause the processing circuitry to receive at least one answer via the peripheral device and determine, based on a combination of the sensor data and the at least one answer, a risk-level of the patient's health associated with at least one of infection, stroke, sepsis, heart failure, COPD, heart failure decompensation, cardiac arrhythmia, or myocardial infarction. The summary is intended to provide an overview of the subject matter described in this disclosure. It is not intended to provide an exclusive or exhaustive explanation of the systems, device, and methods described in detail within the accompanying drawings and description below. Further details of at least one examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example monitoring system, in accordance with at least one technique disclosed herein.

FIG. 2 is a functional block diagram illustrating an example configuration of an example peripheral device of the system of FIG. 1, in accordance with at least one technique disclosed herein.

FIG. 3 is a flowchart illustrating an example method of monitoring a patient in accordance with at least one technique disclosed herein.

FIG. 4 is a flowchart illustrating an example method of assessing a risk-level of the patient's health in accordance with at least one technique disclosed herein.

FIG. 5 is a flowchart illustrating an example decision tree algorithm to assess patient health risk in accordance with at least one technique disclosed herein.

Like reference characters denote like elements throughout the description and figures.

### DETAILED DESCRIPTION

Techniques in this disclosure are directed toward determining a risk-level of a patient's health based on a combination of data from a medical device and user-reported data from a peripheral device. Examples of this disclosure detail techniques that may be implemented in a system comprising a medical device, a peripheral device configured to wirelessly communicate with the medical device, and processing circuitry.

The medical device may be an IMD, such as a cardiac monitor, a cardiac therapy device, a pacemaker, or another cardiac rhythm and/or heart failure (CRHF) device. The medical device may be configured to sense physiological parameter values, such as temperatures, electrocardiogram (ECG) data, impedance, fluid levels, respiration rate, posture, frequency and duration of activities, heart sounds (e.g., from an accelerometer), perfusion (e.g., from optical sensors), and/or the like, which may be indicative of, for example, heart rate, atrial fibrillation (AF), arrhythmia episodes, and/or other cardiac or pulmonary conditions. Each of the parameter values may be an average of a series of sensor data values or other values derived from sensor signals over a certain duration (e.g., an hour, a day, a month, etc.).

The peripheral device may be an external device, such as a smartphone, tablet, computer, or another device that may or may not have a display. The peripheral device may output at least one question to the user (e.g., patient, physician, technician, surgeon, electrophysiologist, clinician (e.g., implanting clinician), caregiver, etc.). As used herein, a question is any communication (e.g., a word, a phrase, a sentence, etc.) expressed so as to elicit information. For example, a question may prompt the user to provide user-reported data in the form of at least one answer. As another example, a question may be an instruction for the user to measure a physiological parameter value and input the physiological parameter value into the peripheral device.

The peripheral device may output for display at least one question to the user to prompt the user to provide user-reported data in the form of at least one answer. In another example, the peripheral device may output a sound indicative of at least one question to the user to prompt the patient to provide user-reported data in the form of at least one answer. The at least one question may relate to the sensor data collected by the medical device. Additionally or alternatively, the at least one question may relate to symptoms of an illness or recent activities. That is, the peripheral device may output the at least one question based on an evaluation of the sensor data.

In some examples, the peripheral device may prompt the user to collect sensor data about the patient's physiological parameters using one or more peripheral devices. For example, the peripheral device may prompt the user to use another peripheral device (e.g., measuring peripheral device), such as a blood pressure cuff, a finger pulse oximeter, a glucose meter, and/or the like to collect sensor data about the patient's blood pressure, proportion of oxygenated hemoglobin, glucose levels, and/or the like, respectively.

Processing circuitry may execute an algorithm to determine whether the sensor data is within a range of pre-determined values, which may be indicative of normal health, or is outside the range of pre-determined values, which may be indicative of a clinically significant event. If the processing circuitry determines, based on the algorithm, that the sensor data are outside the range of pre-determined values, the peripheral device may administer at least one question to the patient or other user. The at least one question may be directly related to characteristics of the sensor data that were outside of the range of pre-determined values. For example, if the processing circuitry determines, based on the algorithm, that the sensor data relating to temperature is outside the range of pre-determined values, which may be indicative of a fever, the peripheral device may administer at least one question to the patient prompting the patient to confirm or deny other symptoms of a fever. The at least one answer to the at least one question may enhance the utility of the sensor data for monitoring the health of the patient by corroborating the characteristics of the sensor data, adding specificity to the sensor data, or identifying false positives in the sensor data.

The sensor data and the at least one answer to the at least one question may be inputted to a decision tree algorithm. According to the decision tree algorithm, various features of the sensor data and various answers may be compared, e.g., in a particular sequence defined by the branching of the decision tree, to various criteria. The results of the comparisons may lead to one of a plurality of possible results for the algorithm as a whole, such as an indication of one of a plurality of conditions of the patient, or one of a plurality of a likelihoods or risk levels of a particular patient condition. In the event that the decision tree algorithm suggests a high risk-level of the patient's health, the peripheral device may begin monitoring the patient more frequently, such as by displaying at least one question to the patient more frequently and/or encouraging the patient to contact a clinician. If the decision tree algorithm suggests a normal or low risk level of the patient's health, the peripheral device may prompt the individual to take no action.

As an example of the operation of the decision tree algorithm, if the patient inputs as an answer that the patient is male, the decision tree algorithm may use thresholds for physiological parameters (e.g., heart rate, blood pressure, weight, etc.) associated with males to determine the risk level of the patient's health. Further, the decision tree algorithm may cause the processing circuitry to output one or more questions to the patient that are intended for male patients, in this way soliciting more particularized and relevant medical information. Similarly, if the patient inputs an answer that the patient is female, the decision tree algorithm may determine the risk level of the patient's health by using thresholds for physiological parameters associated with females and/or causing the processing circuitry to output one or more questions to the patient that are intended for female patients.

As another example, if the data (e.g., sensor data, user-reported data, etc.) indicates that the patient is experiencing a particular medical issue (e.g., heart failure), the decision tree algorithm may cause the processing circuitry to output one or more questions (e.g., whether the patient is experiencing chest pain, shortness of breath, etc.) related to that particular medical issue. In some examples, these questions may request that the patient measure the particular physiological parameters (e.g., blood pressure, heart rate, etc.) related to the particular medical issue for determining the risk-level of the patient's health. Additionally or alternatively, the processing circuitry may cause one or more particular sensors (e.g., the medical device, a blood pressure cuff, a finger pulse oximeter, a glucose meter, etc.) to measure (e.g., continuously or periodically) the particular physiological parameters related to the particular medical issue.

In some examples, data besides the sensor data from medical device and the at least one answer to the at least one question may be inputted to the decision tree algorithm. For example, sensor data collected by one or more measuring peripheral devices (e.g., blood pressure cuff, finger pulse oximeter, glucose meter, etc.) may also be inputted to the decision tree algorithm. In another example, location information from one or more peripheral devices (e.g., a smartphone, tablet, etc.) may also be inputted to the decision tree algorithm. For example, location information collected from a global positioning system (GPS), network (e.g., WIFI, cellular, etc.) data, and/or the like may be inputted to the decision tree algorithm. In yet another example, image, video, and audio data (e.g., from the peripheral device) may also be inputted to the decision tree algorithm. For example, a picture (e.g., of the patient's physical appearance) captured using a camera of the peripheral device and/or an audio recording (e.g., of a cough or breathing quality) recorded using a microphone of the peripheral device may be inputted to the decision tree algorithm. The decision tree algorithm may then use this additional data to, at least in part, suggest the risk-level of the patient's health.

In some examples, the peripheral device may schedule the display of at least one questions to the patient. For example, the peripheral device may display a first question to the patient a first pre-determined amount of time (e.g., 24 hours) after the decision tree algorithm determines a risk-level of the patient's health, irrespective of whether the decision tree algorithm suggests a high risk-level of the patient's health. The peripheral device may then display a second question to the patient a second pre-determined amount of time (e.g., 48 hours) after the decision tree algorithm determines a risk-level of the patient's health, and so on.

Processing circuitry (e.g., of the peripheral device) may determine a probability of a patient experiencing a medical issue, such as a heart failure. For example, the processing circuitry may use a Bayesian Belief Network (BBN) framework or any other suitable technique for identifying when the patient is at risk. In some examples, sensor data (e.g., collected by the medical device, one or more peripheral devices, including measuring peripheral devices, etc.) relating to various physiological parameters may represent input data for the BBN framework. The physiological parameters may include intra-thoracic impedance (IMP), atrial fibrillation (AF) burden and rate control information, night heart rate, heart rate variability, patient activity, and/or the like. Thus, in this example, based on the input data, the BBN framework may output a probability of the patient experiencing heart failure.

In some examples, the processing circuitry may determine the probability of the patient experiencing the medical issue by comparing the physiological parameter values to corresponding thresholds. For example, the processing circuitry may assign a score to each physiological parameter based on the extent to which the physiological parameter value (e.g., IMP) is above or below the corresponding threshold (e.g., an IMP threshold). The processing circuitry may then add the scores for the various physiological parameters to determine (e.g., via the BBN framework) the probability of the patient experiencing a medical issue (e.g., heart failure).

The peripheral device may output a question based on the probability of the patient experiencing a medical issue, such as heart failure. That is, the peripheral device may output the at least one question for the patient to answer in response to the probability of the patient experiencing the medical issue exceeding a threshold probability. The threshold probability may indicate a significant risk of an upcoming medical issue (e.g., an imminent heart failure) or exacerbation thereof (e.g., worsening heart failure). In some examples, the peripheral device may output a question based on a physiological parameter satisfying a respective threshold. In such examples, the answer may influence the determination of the risk of the medical issue, such as by processing circuitry changing one or more thresholds or other criteria based on the answer.

In any case, the medical device and the one or more peripheral devices, including the measuring peripheral devices, may collect data (e.g., sensor data, user-reported data, etc.) continuously or periodically (e.g., hourly, daily, monthly, etc.). In this way, the processing circuitry may continuously or periodically redetermine the risk-level of the patient's health, the probability of the patient experiencing a medical issue, and so on. The use of combined data streams from medical device sensors and the answers may advantageously provide a more holistic and complete picture of patient status. Triggering the presentation of the questions based on an analysis of sensor data may allow the questions to be advantageously posed at a relevant time.

In some examples, the decision tree algorithm may accept time-series data from the IMD and may alert the patient to a high-risk change in the time-series data. For example, the decision tree algorithm may determine an increased or supra-threshold risk of stroke based on a change in a CHADS₂ and CHA₂DS₂-VASc score (e.g., clinical prediction rules for estimating the risk of stroke, such as in patients with non-rheumatic AF, a common and serious heart arrhythmia associated with thromboembolic stroke) or other stroke risk score of the patient over time. The peripheral device may request patient information from the patient, caregiver, or a healthcare organization (e.g., from electronic medical record (EMR) system) to determine, at least in part, a risk-level of the patient's health (e.g., the CHA₂DS₂-VASc score). In any case, the algorithm may determine that the stroke risk score has changed based on, among other things, changes to heart failure symptoms and/or atrial arrhythmia burden.

In some examples, the IMD may process the sensor data using the algorithm and send at least a portion of the processed sensor data to the peripheral device. In some examples, the IMD may implement the portions of the algorithm associated with determining whether the sensor data is out of range or otherwise satisfies one or more criteria for the peripheral device to ask questions of the patient. In other examples, the peripheral device may receive the sensor data directly from the IMD, and the peripheral device may process the sensor data using the algorithm.

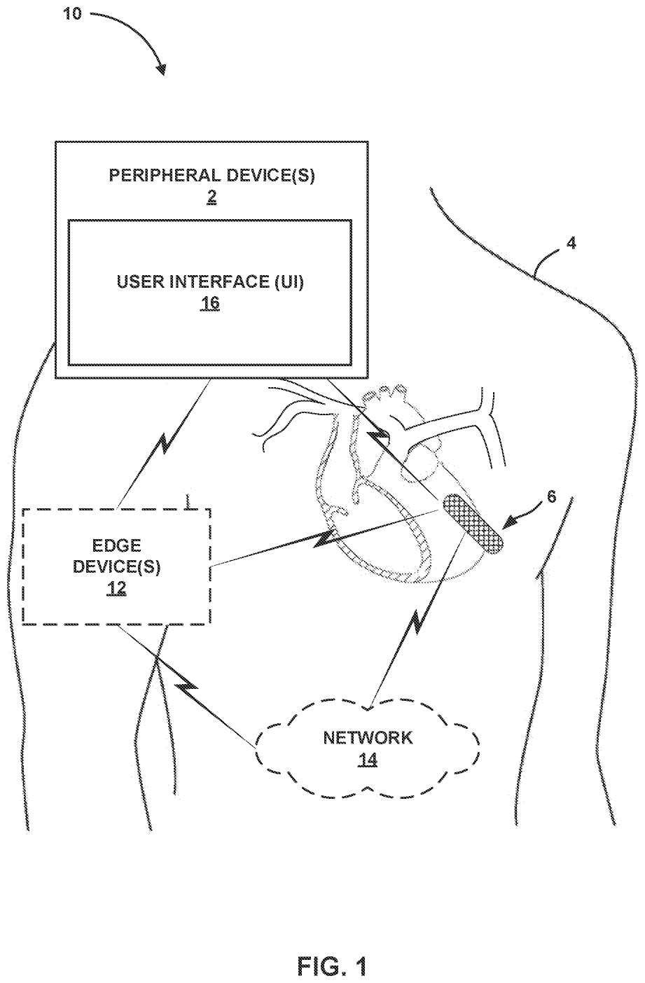

FIG. 1 illustrates the environment of an example monitoring system 10 in conjunction with a patient 4. In some examples, system 10 may implement the various patient monitoring techniques disclosed herein. System 10 includes at least one medical device 6 and at least one peripheral device 2. While medical device 6 in some instances includes an IMD, as shown in FIG. 1, the techniques of this disclosure are not so limited. For illustrative purposes, however, medical device 6 may in some instances be referred to herein simply as IMD 6 or IMD(s) 6.

Peripheral device 2 may be referred to in some instances herein as a plurality of “peripheral device(s) 2,” while in other instances may be referred to simply as “peripheral device 2,” where appropriate. Peripheral device 2 may be a computing device with a display viewable by a patient 4 or another user. The user may be a patient, physician, technician, surgeon, electrophysiologist, clinician (e.g., implanting clinician), caregiver, etc. Patient 4 ordinarily, but not necessarily, will be a human.

System 10 may be implemented in any setting where at least one peripheral device 2 may interface with and/or monitor at least one medical device 6. Peripheral device 2 may interface with and/or monitor medical device 6, for example, by pairing with the medical device 6. Peripheral device 2 and medical device 6 may communicate wirelessly, e.g., according to a Bluetooth® standard.

Peripheral device 2 may obtain data from medical device 6. The data may include historical data stored to memory of medical device 6, and/or real-time data collected by medical device 6. Example types of data include battery capacity or other performance data of the medical device 6, and sensor data collected by the medical device. Sensor data may include bioimpedance, patient temperature, ECG data, blood oxygen data, activity, and other data about physiological parameters. In some examples, peripheral device 2 may obtain sensor data (e.g., data about the physiological parameters) collected by the medical device 6, such as physiological parameter values, physiological parameter waveforms, physiological parameter labels (e.g., ‘abnormal ECG detected’) and other data about physiological parameters.

In other examples, peripheral device 2 may obtain sensor data using sensors on peripheral device 2 (e.g., a camera, a microphone, a blood pressure sensor, etc.), or from one or more measuring peripheral devices (e.g., a blood pressure cuff, a finger pulse oximeter, a glucose meter, etc.). For example, peripheral device 2 may prompt patient 4 to use a blood pressure cuff to measure the blood pressure of patient 4. Patient 4, a caregiver, or some other user may then input the blood pressure measurement into peripheral device 2. In some examples, peripheral device 2 may also obtain patient information from the patient, caregiver, or a healthcare organization (e.g., from an EMR system) to determine, at least in part, the risk-level of patient's health (e.g., the CHA₂DS₂-VASc score). The patient information may include, but is not limited to, clinical history, prescriptions, examinations, vaccination status, medical procedures, and/or the like.

In some examples, peripheral device 2 may evaluate the sensor data and, based on the evaluation of the sensor data, output at least one question related to the sensor data collected by the medical device 6 for patient 4 or another user (e.g., physician, technician, caregiver, etc.) to answer. For example, if the physiological parameter values are outside of an expected range or otherwise satisfy one or more criteria, and are therefore indicative of a clinically significant event or change in patient condition, peripheral device 2 may output at least one question (e.g., by displaying at least one question via a user interface 16 (“UI 16”) of the peripheral device 2), where the purpose of at least one question may be to diagnose and/or triage patient 4. In some examples, patient 4 may see a risk-level of the patient's health and updates thereto via UI 16 of peripheral device 2.

In some examples, patient 4 may enter at least one answer to the question via the same peripheral device 2 that outputted the at least one question. In other examples, patient 4 may enter at least one answer to the question via a different peripheral device 2 that may then transfer data, including data about the at least one answer, to the peripheral device 2 that outputted the at least one question. In such examples, and in other examples, network 14 or edge device(s) 12 may facilitate the exchange of data between the at least one medical device 6 and the at least one peripheral device 2.

In some examples, system 10 may determine, based on a combination of the sensor data and the at least one answer, a risk-level of the patient's health associated with clinically significant events. For example, system 10 may determine a risk-level of the patient's health associated with at least one of infection, stroke, sepsis, heart failure, chronic obstructive pulmonary disease (COPD), heart failure decompensation, COPD exacerbation, cardiac arrhythmia, or myocardial infarction based on a combination of sensor data (e.g., from medical device 6, peripheral device 2, one or more other peripheral devices, etc.) for physiological parameters (e.g., temperature, ECG data, oxygen saturation, blood pressure, activity level, caloric expenditure, etc.) and the at least one answer to the at least one question outputted by system 10. In some examples, system 10 may also determine a risk-level of the patient's health associated with a pandemic infection (e.g., COVID-19), cancer, and other conditions. System 10 may determine the risk-level of the patient's health based on additional data, such as patient information from an EMR system.

In some examples, peripheral device 2 may include at least one of a cellular phone, a ‘smartphone,’ a satellite phone, a notebook computer, a tablet computer, a wearable device, a computer workstation, at least one server, a personal digital assistant, a handheld computing device, a virtual reality headset, wireless access points, motion or presence sensor devices, or any other computing device that may run an application that enables the peripheral device 2 to interact with medical device 6 or interact with another peripheral device 2 that is, in turn, configured to interact with medical device 6.

Peripheral device 2 may be configured to communicate with medical device 6 via wired or wireless communication. Peripheral device 2, for example, may communicate via near-field communication (NFC) technologies (e.g., inductive coupling, NFC or other communication technologies operable at ranges less than 10-20 cm) and/or far-field communication technologies (e.g., Radio Frequency (RF) telemetry according to the 802.11, Bluetooth® specification sets, or other communication technologies operable at ranges greater than NFC technologies).

Peripheral device 2 may include UI 16. In some examples, UI 16 may be a graphical user interface (GUI), an interactive UI, etc. In some examples, UI 16 may further include a command line interface. In some examples, peripheral device 2 and/or edge device(s) 12 may include a display system (not shown). In such examples, the display system may include system software for generating UI data to be presented for display and/or interaction. In some examples, processing circuitry, such as that of peripheral device 2, may receive UI data from another device, such as from one of edge device(s) 12 or servers, that peripheral device 2 may use to generate UI data to be presented for display and/or interaction.

Peripheral device 2 may be configured to receive, via UI 16, input from the user. In some examples, UI 16 may include a display (e.g., a liquid crystal display (LCD) or light emitting diode (LED) display). In some examples, a display of peripheral device 2 may be a touch screen display, and a user may interact with peripheral device 2 via the touch screen display. It should be noted that the user may also interact with peripheral device 2 remotely via a network computing device.

In some examples, UI 16 may include a keypad. In some examples, UI 16 may include the keypad and the display. The keypad may take the form of an alphanumeric keypad or a reduced set of keys associated with particular functions. Peripheral device 2 may additionally or alternatively include a peripheral pointing device, such as a mouse, via which the user may interact with UI 16. In some examples, UI 16 may include a UI that utilizes virtual reality (VR), augmented reality (AR), or mixed reality (MR) Uls, such as those that may be implemented via a VR, AR, or MR headset.

In some examples, system 10 may include at least one processor and at least one storage device. In some examples, the at least one storage device may include a memory, such as a memory device of peripheral device 2, where the memory may be configured to store sensed data collected from IMD 6, and answers to the at least one question administered. In some examples, the storage devices may be configured to store data, such as physiological parameter values, patient-status updates, device interrogation data, etc. The at least one processor, may be in communication with the at least one of the storage device configured to store patient data.

Peripheral device 2 may determine a set of data items. The set of data items may include at least one of: data (e.g., sensor data) obtained from medical device 6, or user-reported data. In such examples, peripheral device 2 may determine, based at least in part on the set of data items, an abnormality, such as sensor data for patient 4 that are outside of the range of pre-determined values. In some examples, the set of data items may include data about a potential infection, heart failure events, abnormal physiological parameters, or another medical condition. Such data may include physiological parameter values, physiological parameter waveforms, physiological parameter labels, user-reported data, etc., corresponding to each medical condition. In any case, system 10 may determine the set of data items to determine a risk-level of the patient's health associated with at least one of infection or stroke in accordance with the techniques disclosed herein.

Medical device 6 may be implanted outside of a thoracic cavity of patient 4 (e.g., subcutaneously in the pectoral location illustrated in FIG. 1). In some examples, medical device 6 may be positioned near the sternum near or just below the level of the heart of patient 4, e.g., at least partially within the cardiac silhouette. An 1MB 6 may include, be, or be part of a variety of devices or integrated systems, such as implantable cardiac monitors (ICMs), implantable pacemakers, including those that deliver cardiac resynchronization therapy (CRT), implantable cardioverter-defibrillators (ICDs), diagnostic devices, cardiac devices, neuromodulation device, etc. In some examples, the techniques of this disclosure may be implemented in medical devices other than CIEDs, such as spinal cord stimulators, deep brain stimulators, gastrological stimulators, urological stimulators, other neurostimulators, orthopedic implants, respiratory monitoring implants, etc.

In some examples, medical device 6 may include at least one CIED. In some examples, patient 4 may interface with multiple medical devices 6, concurrently. In some examples, patient 4 may have multiple IMDs 6 implanted within the body of patient 4. In some examples, medical device 6 may include a combination of at least one implanted and/or non-implanted medical devices 6. An example of a non-implanted medical device 6 includes a wearable device (e.g., monitoring watch, wearable defibrillator, heart monitor, a blood pressure cuff, a finger pulse oximeter, a glucose meter, etc.) or any other external medical devices 6 (e.g., a weight scale) configured to obtain physiological data of patient 4.

In some examples, medical device 6 may include diagnostic medical devices. For example, medical device 6 may include a device that diagnoses or predicts heart failure events or that detects worsening heart failure of patient 4. In any case, medical device 6 may be configured to determine a health status relating to patient 4. Medical device 6 may transmit the diagnosis or health status to peripheral device 2, so that peripheral device 2 may correlate the diagnosis or health status to determine whether patient 4 is experiencing a clinically significant event (e.g., an infection, a fever, AF, stroke, etc.).

In some examples, medical device 6 may operate as a therapy delivery device, such as an implantable pacemaker, a cardioverter and/or defibrillator, a drug delivery device that delivers therapeutic substances to patient 4 via at least one catheters, or as a combination therapy device that delivers both electrical signals and therapeutic substances. For example, medical device(s) may deliver electrical signals to the heart of patient 4.

In addition, while certain example medical devices are described as being insertable or implantable devices, the techniques of this disclosure are not so limited, and persons skilled in the art will understand that the techniques of this disclosure may be implemented with medical devices that are not configured to be insertable or implantable, such as wearable devices or other external medical devices. In a non-limiting example, medical device 6 may include wearable devices (e.g., smart watches, headsets, etc.) configured to obtain physiological data (e.g., activity data, heart rate, etc.) and transfer such data to peripheral device 2, network 14, edge device(s) 12, etc. for subsequent utilization, in accordance with at least one of the various techniques of this disclosure.

Moreover, while certain example medical devices are described as being electrical devices or electrically-active devices, the techniques of this disclosure are not so limited, and person skilled in the art will understand that, in some examples, medical device 6 may include non-electrical or non-electrically-active devices (e.g., orthopedic implants, etc.). In some examples, medical device 6 takes the form of the Reveal LINQ™ Insertable Cardiac Monitor (ICM), or another ICM similar to, e.g., a version or modification of, the LINQTM ICM, developed by Medtronic, Inc., of Minneapolis, MN. In such examples, medical device 6 may facilitate relatively longer-term monitoring of patients during normal daily activities.

In some examples, system 10 may be implemented in a setting that includes network 14 and/or edge device(s) 12. That is, in some examples, system 10 may operate in the context of network 14 and/or include at least one edge device(s) 12. In some examples, network 14 may include edge device(s) 12. Similarly, peripheral device 2 may include functionality of edge device(s) 12 and thus, may also serve as one of edge device(s) 12.

In some examples, edge device(s) 12 include modems, routers, Internet of Things (IoT) devices or systems, smart speakers, screen-enhanced smart speakers, personal assistant devices, etc. In some examples, edge device(s) 12 may include user-facing or client devices, such as smartphones, tablet computers, personal digital assistants (PDAs), and other mobile computing devices.

In examples involving network 14 and/or edge device(s) 12, system 10 may be implemented in a home setting, a hospital setting, or in any setting comprising network 14 and/or edge device(s) 12. The example techniques may be used with medical device 6, which may be in wireless communication with at least one edge device(s) 12 and other devices not pictured in FIG. 1 (e.g., network servers).

In some examples, peripheral device 2 may be configured to communicate with at least one of medical device 6, edge device(s) 12, or network 14 operating a network service such as the Medtronic CareLink® Network developed by Medtronic, Inc., of Minneapolis, Minn. In some examples, medical device 6 may communicate, via Bluetooth®, with peripheral device 2. In some instances, network 14 may include at least one of edge device(s) 12. Network 14 may be and/or include any appropriate network, including a private network, a personal area network, an intranet, a local area network (LAN), a wide area network, a cable network, a satellite network, a cellular network, a peer-to-peer network, a global network (e.g., the Internet), a cloud network, an edge network, a network of Bluetooth® devices, etc., or a combination thereof, some or all of which may or may not have access to and/or from the Internet. That is, in some examples, network 14 includes the Internet. In an illustrative example, peripheral device 2 may periodically transmit and/or receive various data items, via network 14, to and/or from one of medical device 6, and/or edge device(s) 12.

In some examples, peripheral device 2 may be configured to retrieve data from medical device 6. The retrieved data may include physiological parameter values sensed (e.g., measured) by medical device 6, indications of episodes of arrhythmia or other maladies detected by medical device 6, and physiological signals obtained by medical device 6. In some examples, peripheral device 2 may retrieve cardiac EGM segments recorded by peripheral device 2, e.g., due to peripheral device 2 determining that an episode of arrhythmia or another malady occurred during the segment, or in response to a request, from patient 4 or another user, to record the segment.

In some examples, the user may also use peripheral device 2 to retrieve information from medical device 6 regarding other sensed physiological parameters of patient 4, such as activity, temperature or posture. In some examples, edge device(s) 12 may interact with medical device 6 in a manner similar to peripheral device 2 (e.g., to program medical device 6 and/or retrieve data from medical device 6).

Processing circuitry of system 10, e.g., of medical device 6, peripheral device 2, edge device(s) 12, and/or of at least one other computing devices (e.g., remote servers), may be configured to perform the example techniques of this disclosure for determining an actionable data of patient 4. In some examples, processing circuitry of system 10 obtains physiological parameter values, medical device diagnostics, etc. to determine whether to display at least one question to patient 4 and/or notify patient to contact a HCP.

In some examples, processing circuitry of system 10 (e.g., of peripheral device 2) may output at least one physiological and/or psychological question to patient 4 when data obtained from medical device (e.g. medical device diagnostics, sensor data, etc.) and/or other user-reported data indicate the onset of a patient abnormality. In some examples, processing circuitry of system 10 may notify patient 4, such as by causing medical device 6 and/or peripheral device 2 to generate an audible alert, a visual alert, a tactile alert (e.g., a vibration or vibrational pattern), a text prompt, and/or a button prompt. Additionally or alternatively, the notification or some variation thereof may be provided to other devices, e.g., via network 14 and to recipients other than patient 4 (e.g., a HCP). Several different levels of alerts may be used based on the risk-level of the patient's health (e.g., potential infection) detected in accordance with at least one of the various techniques disclosed herein.

In some examples, peripheral device 2 may schedule the display of at least one questions to the patient. For example, peripheral device 2 may display a first question to the patient a first pre-determined amount of time (e.g., 24 hours) after the decision tree algorithm determines a risk-level of the patient's health, irrespective of whether the decision tree algorithm suggests a high risk-level of the patient's health. Peripheral device 2 may then display a second question to the patient a second pre-determined amount of time (e.g., 48 hours) after the decision tree algorithm determines a risk-level of the patient's health, and so on.

In some examples, processing circuitry of system 10, e.g., of peripheral device 2, provides an alert to patient 4 and/or other users (e.g., physician, technician, caregiver, etc.) when a combination of user-reported data (e.g., answers to physiological and/or psychological questions, etc.) and medical device diagnostic data indicate the onset of an abnormality. The process for determining when to alert patient 4 involves measuring an abnormality (e.g., severity or probability levels) against at least one threshold values. The alert may be an audible alert generated by medical device 6 and/or peripheral device 2, a visual alert generated by peripheral device 2, such as a text prompt or flashing buttons or screen, or a tactile alert generated by medical device 6 and/or peripheral device 2 such as a vibration or vibrational pattern. Furthermore, the alert may be provided to other devices, e.g., via network 14. Several different levels of alerts may be used based on a severity of a potential clinical event.

In some examples, medical device 6 may be an IMD 6, such as a pacemaker and other CRHF devices, that has at least one sensor onboard (e.g., a temperature sensor, an ECG sensor, etc.) that may track a patient's health status. System 10 may combine data sensed by IMD 6 with data reported by patient 4, such as at least one answer to at least one question displayed to patient 4, into an application or algorithm on a peripheral device 2 to guide further actions by patient 4 and/or HCP and/or clinician. In the latter two situations, the data may be pushed to the HCP and/or clinician after patient 4 has interacted with the application on peripheral device 2 and answered the at least one question displayed to patient 4.

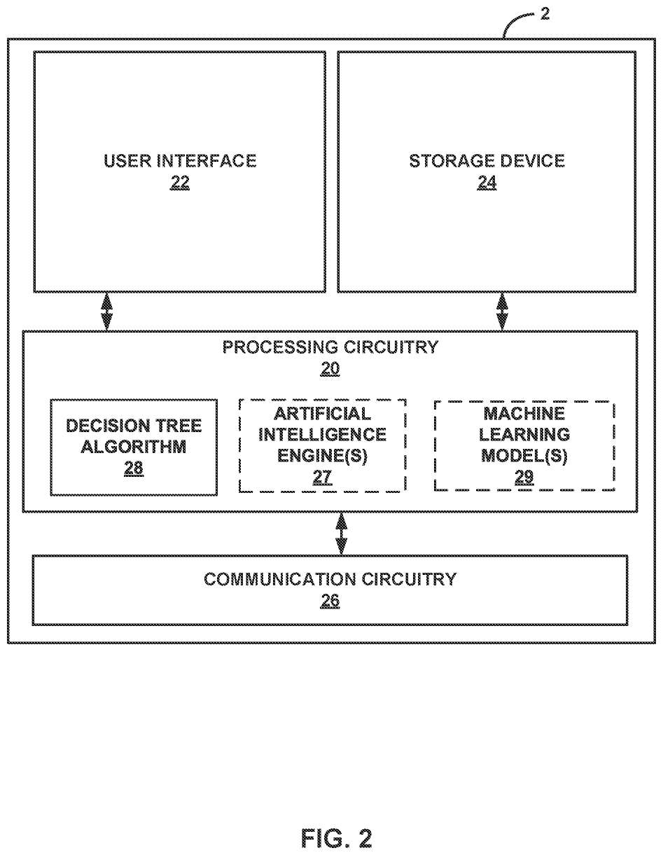

FIG. 2 is a block diagram illustrating an example configuration of components of peripheral device 2, which may generally take the form of a computing device. In the example of FIG. 2, the peripheral device 2 includes processing circuitry 20, communication circuitry 26, storage device 24, and UI 16.

Processing circuitry 20 may include one or more processors that are configured to implement functionality and/or process instructions for execution within peripheral device 2. For example, processing circuitry 20 may be capable of processing instructions stored in storage device 24. Processing circuitry 20 may include, for example, microprocessors, a digital signal processors (DSPs), an application specific integrated circuits (ASICs), field programmable gate arrays (FPGAs), complex programmable logic devices (CPLDs), or equivalent integrated or discrete logic circuitry, or a combination of any of the foregoing devices or circuitry. Accordingly, processing circuitry 20 may include any suitable structure, whether in hardware, software, firmware, or any combination thereof, to perform the functions ascribed herein to processing circuitry 20.

In some examples, processing circuitry 20 may process and analyze the sensor data using a scoring system. That is, points indicative of a risk-level of patient's health may be assigned to various types of sensor data and answers to questions, and processing circuitry 20 may add the points to determine a risk-level of patient's health for a variety of conditions. For example, a normal heart rate (e.g., 70 beats per minute (BPM) may be assigned 0 points, and a high heart rate (e.g., 90 BPM) may be assigned 10 points. Points may be assigned in a similar manner to blood pressure, glucose levels, temperature, and/or the like, and processing circuitry 20 may add the scores to determine a risk-level for a variety of conditions (e.g., infection, stroke, sepsis, heart failure, COPD, heart failure decompensation, COPD exacerbation, cardiac arrhythmia, myocardial infarction, etc.).

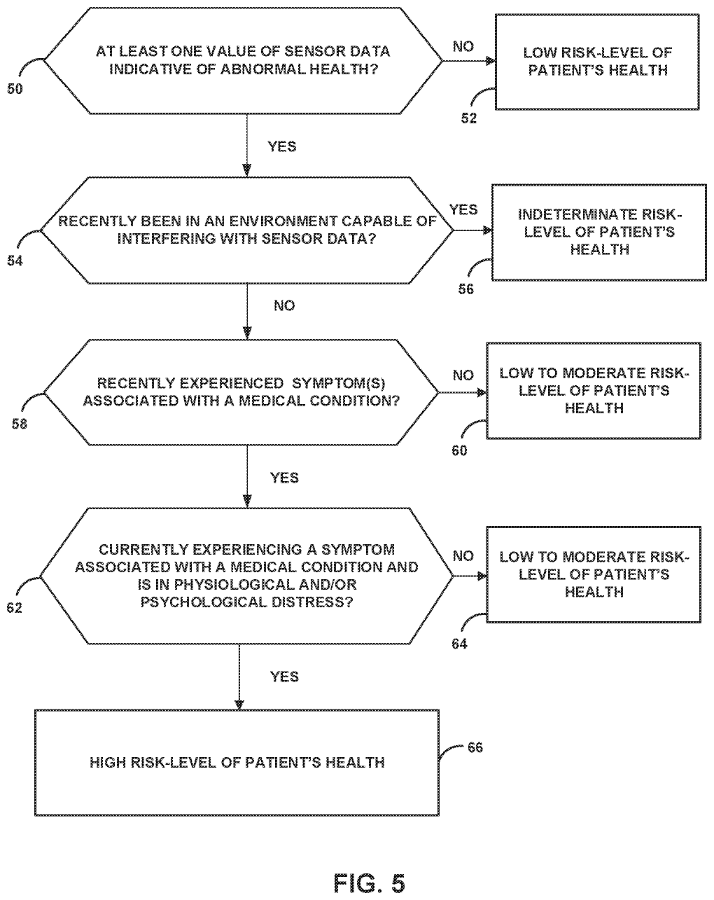

In some examples, processing circuitry 20 may use a trained machine learning (ML) model 29 and/or an artificial intelligence (AI) engine 27. Trained ML model 29 and/or AI engine 27 may be configured to process and analyze the sensor data from medical device 6 and user input in response to questions in accordance with certain examples of this disclosure where ML models are considered advantageous (e.g., predictive modeling, inference detection, contextual matching, natural language processing, etc.). Examples of ML models and/or AI engines that may be so configured to perform aspects of this disclosure include classifiers and non-classification ML models, artificial neural networks (“NNs”), linear regression models, logistic regression models, decision trees, support vector machines (“SVM”), Naïve or a non-Naïve Bayes network, k-nearest neighbors (“KNN”) models, deep learning (DL) models, k-means models, clustering models, random forest models, or any combination thereof. Depending on the implementation, the ML models may be supervised, unsupervised or in some instances, a hybrid combination (e.g., semi supervised). These models may be trained based on data indicating how users (e.g., patient 4) interact with peripheral device 2. Additionally or alternatively, these models may be trained based on training sets of physiological parameter data. In the illustrated example, processing circuitry 20 implements a decision tree algorithm 28, e.g., as discussed with respect to FIG. 5, but may additionally or alternatively implement any ML model 29 and/or AI engine 27 in some examples.

Communication circuitry 26 may include any suitable hardware, firmware, software or any combination thereof for communicating with another device, such as medical device(s) 6. Under the control of processing circuitry 20, communication circuitry 26 may receive downlink telemetry from, as well as send uplink telemetry to, medical device(s) 6, or another device. Communication circuitry 26 may be configured to transmit or receive signals via inductive coupling, electromagnetic coupling, NFC, RF communication, Bluetooth®, Wi Fi™, or other proprietary or non-proprietary wireless communication schemes. Communication circuitry 26 may also be configured to communicate with devices other than medical device(s) 6 via any of a variety of forms of wired and/or wireless communication and/or network protocols. In some examples, peripheral device 2 may perform telemetry selection through a sweep (e.g., TelC, TelB, TelM, Bluetooth®, etc.).

Storage device 24 may be configured to store information within peripheral device(s) 2 during operation. Storage device 24 may include a computer-readable storage medium or computer-readable storage device. In some examples, storage device 24 includes one or more of a short-term memory or a long-term memory. Storage device 24 may include, for example, read-only memory (ROM), random access memory (RAM), non-volatile RAM (NVRAM), Dynamic RAM (DRAM), Static RAM (SRAM), magnetic discs, optical discs, flash memory, forms of electrically erasable programmable ROM (EEPROM) or erasable programmable ROM (EPROM), or any other digital media.

In some examples, storage device 24 is used to store data indicative of instructions for execution by processing circuitry 20. Storage device 24 may be used by software or applications running on peripheral device 2 to temporarily store information during program execution. Storage device 24 may also store historical medical device data, historical patient data, number of days since a particular physiological parameter has been above or below a certain threshold, etc.), AI and/or ML training sets, etc. In some examples, data collected (e.g., directly or indirectly) by peripheral device 2 may be used to further train ML model 29 and/or AI engine 27.

Data exchanged between peripheral device 2, edge device(s) 12, network 14, and medical device(s) 6 may include operational parameters of medical device(s) 6. Peripheral device(s) 2 may transmit data, including computer readable instructions, to medical device(s) 6. Medical device(s) 6 may receive and implement the computer readable instructions. In some examples, the computer readable instructions, when implemented by medical device(s) 6, may control medical device(s) 6 to change one or more operational parameters, export collected data, etc. In an illustrative example, processing circuitry 20 may transmit an instruction to medical device(s) 6 which requests medical device(s) 6 to export collected data (e.g., temperature, ECGs, etc.) to peripheral device(s) 2, edge device(s) 12, and/or network 14. In turn, peripheral device(s) 2, edge device(s) 12, and/or network 14 may receive the collected data from medical device(s) 6 and store the collected data, for example, in storage device 24. In addition, processing circuitry 20 may transmit an instruction to medical device(s) 6 which requests medical device(s) 6 to export operational parameters (e.g., battery, impedance, pulse width, pacing %, etc.).

In the example illustrated in FIG. 2, processing circuitry 20 is configured to perform the various techniques described herein. To avoid confusion, processing circuitry 20 is described as performing the various processing techniques prescribed to peripheral device(s) 2, but it should be understood that at least some of these techniques may also be performed by other processing circuitry (e.g., processing circuitry of medical device(s) 6, processing circuitry of server(s), processing circuitry of edge device(s) 12, etc.). For example, processing circuitry of medical device 6 may determine, based on a combination of the sensor data and the at least one answer, a risk-level of the patient's health associated with at least one of various medical conditions.

In accordance with techniques of this disclosure, processing circuitry 20 (e.g., of peripheral device 2) may determine a probability of a patient experiencing a medical issue, such as a heart failure. For example, processing circuitry 20 may use a BBN framework or any other suitable technique for identifying when the patient is at risk. In some examples, sensor data (e.g., collected by the medical device, one or more peripheral devices, including measuring peripheral devices, etc.) relating to various physiological parameters may represent input data for the BBN framework. The physiological parameters may include IMP, AF burden and rate control information, night heart rate, heart rate variability, patient activity, and/or the like. Thus, in this example, based on the input data, the BBN framework may output a probability of the patient experiencing heart failure.

In some examples, processing circuitry 20 may determine the probability of patient 4 experiencing the medical issue by comparing the physiological parameter values to corresponding thresholds. For example, processing circuitry 20 may assign a score to each physiological parameter based on the extent to which the physiological parameter value (e.g., IMP) is above or below the corresponding threshold (e.g., an IMP threshold). Processing circuitry 20 may then add the scores for the various physiological parameters to determine (e.g., via the BBN framework) the probability of patient 4 experiencing a medical issue (e.g., heart failure).

Peripheral device 2 may output a question based on the probability of patient 4 experiencing a medical issue, such as heart failure. That is, peripheral device 2 may output the at least one question for patient 4 to answer in response to the probability of patient 4 experiencing the medical issue exceeding a threshold probability. The threshold probability may indicate a significant risk of an upcoming medical issue (e.g., an imminent heart failure) or exacerbation thereof (e.g., worsening heart failure). In some examples, the peripheral device may output a question based on a physiological parameter satisfying a respective threshold. In such examples, the answer may influence the determination of the risk of the medical issue, such as by processing circuitry changing one or more thresholds or other criteria based on the answer.

In any case, medical device 6 and/or peripheral device(s) 2 (e.g., measuring peripheral devices) may collect data (e.g., sensor data, user-reported data, etc.) continuously or periodically (e.g., hourly, daily, monthly, etc.). In this way, processing circuitry 20 may continuously or periodically redetermine the risk-level of the patient's health, the probability of patient 4 experiencing a medical issue, and so on. The use of combined data streams from sensors and the answers may advantageously provide a more holistic and complete picture of patient status. Triggering the presentation of the questions based on an analysis of sensor data may allow the questions to be advantageously posed at a relevant time.

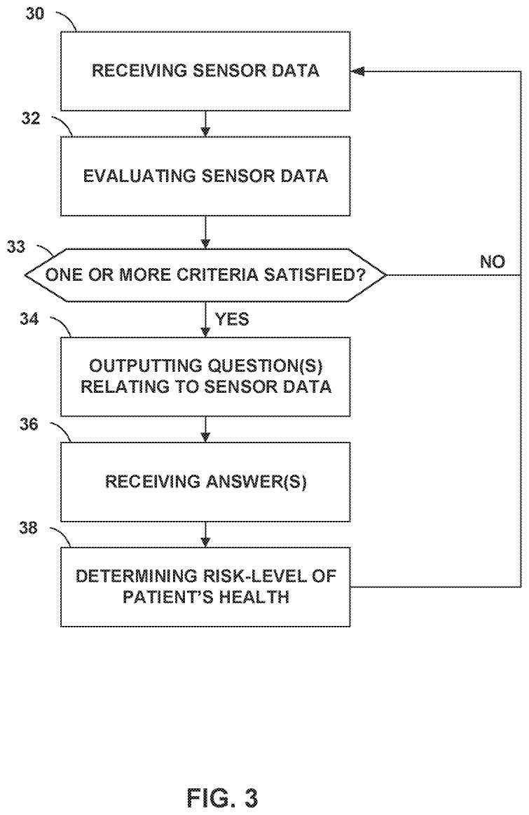

FIG. 3 is a flowchart illustrating an example method of monitoring a patient. The method may include using a system (e.g., system 10) comprising medical device 6, peripheral device 2 configured to wirelessly communicate with medical device 6, and processing circuitry (e.g., processing circuitry 20 of peripheral device 2).

The method may include, by the processing circuitry, receiving sensor data collected by the medical device 6 (30) and evaluating the sensor data (32). Based on the evaluation of the sensor data (32), the processing circuitry may determine whether the sensor data satisfies one or more criteria (33). The criteria may be configured such that their satisfaction indicates a certain likelihood of a clinically significant event, which may merit further investigation.

If the criteria are not satisfied (NO of 33), the processing circuitry may continue to receive and evaluate sensor data (30, 32). If the criteria are satisfied (YES of 33), the processing circuitry may output via the peripheral device 2 at least one question relating to the sensor data collected by the medical device 6 for a patient 4 to answer (34). After receiving at least one answer from patient 4 (36), the method may further include determining, based on a combination of the sensor data and the at least one answer, a risk-level of the patient's health (e.g., associated with at least one of infection or stroke) (38).

In some examples, the sensor data collected by medical device 6 (30) may comprise sensor data measured by medical device 6 (e.g., cardiac monitor, cardiac therapy device). Medical device 6 may be an IMD 6. The sensor data being received may be at least one of temperature data or ECG data. Sensor data may include an average of a series of measurements over time. In some examples, the processing circuitry may be configured to receive the sensor data from medical device 6. For example, processing circuitry 20 of peripheral device 2 may wirelessly receive sensor data collected by medical device 6. Additionally or alternatively, processing circuitry of medical device 6 may receive sensor data collected by the at least one sensor of medical device 6.

In some examples, evaluating the sensor data (32) and determining whether the sensor data satisfies one or more criteria (33) may include determining whether to output a question for patient 4 to answer. For example, a pre-determined range of values for body temperatures may range from 97° F. (36.1° C.) to 99° F. (37.2° C.). In such an example, sensor data indicating that the body temperature of patient 4 is 98° F., which is within the pre-determined upper range 99° F., may not cause the peripheral device to output at least one question relating to the sensor data (e.g., a question relating to body temperature). Alternatively, the peripheral device may still output at least one question relating to the sensor data to confirm the accuracy of the sensor data. The question may be physiological and/or psychological and for the purpose of diagnosing patient 4. For example, the question may ask patient 4 whether the patient recently experienced sweating, chills and shivering, headaches, muscle aches, loss of appetite, irritability, dehydration, and/or general weakness to determine if patient 4 is suffering from an infection, fever, or some other medical condition for which a high body temperature is a symptom.

In some examples, the question may ask whether patient 4 has recently been in an environment capable of interfering with the sensor data (e.g., a very hot environment or a very cold environment, which can interfere with sensor data relating to body temperature). In other examples, the question may ask patient 4 or another user to use one or more sensors to measure environmental conditions. For example, the question may ask a caretaker of patient 4 to use a thermometer to measure the temperature of the area that patient 4 is occupying. Processing circuitry of medical device 6 may then use the data on the environmental conditions in conjunction with the sensor data relating to patient 4 to determine the risk-level of patient 4 in accordance with techniques of this disclosure.

In some examples, determining whether the sensor data satisfies criteria (33) may include determining that at least one value of the sensor data is outside a pre-determined range of values, e.g., is above, below, or otherwise satisfies a threshold that defines the range. In response to determining that the at least one value is outside the pre-determined range, the method may further include outputting at least one question relating to the sensor data. For example, a pre-determined range of values for body temperatures may range from 97° F. (36.1° C.) to 99° F. (37.2° C.). In such an example, sensor data indicating that the body temperature of patient 4 is 100° F., which is above the pre-determined upper range 99° F., may cause the peripheral device to output at least one question relating to the sensor data. The question may be physiological and/or psychological and for the purpose of diagnosing patient 4. For example, the question may ask patient 4 with a body temperature of 100° F. whether the patient recently experienced sweating, chills and shivering, headaches, muscle aches, loss of appetite, irritability, dehydration, and/or general weakness to determine if patient 4 is suffering from an infection, fever, or some other medical condition for which a high body temperature is a symptom. Additionally or alternatively, the question may ask whether patient 4 has recently been in an environment capable of interfering with sensor data (e.g., a very hot environment or a very cold environment).

In some examples, evaluating the sensor data (32) may include confirming the accuracy of the sensor data using the patient's at least one answer (34) to the at least one question. In the event that patient 4 answers that patient 4 recently experienced sweating, chills and shivering, headaches, muscle aches, loss of appetite, irritability, dehydration, and/or general weakness and that patient 4 has not recently been in an environment capable of interfering with sensor data, the accuracy of the sensor data indicating a high body temperature is confirmed, increasing the reliability of the diagnosis of infection, fever, or some other medical condition for which a high body temperature is a symptom.

In some examples, determining a risk-level of the patient's health (38) may be based on a combination of the sensor data and the at least one answer. For example, a pre-determined range of values for body temperatures may range from 97° F. (36.1° C.) to 99° F. (37.2° C.). In such an example, sensor data indicating that the body temperature of patient 4 is 98° F., is within the pre-determined range of 97° F. to 99° F. Additionally, patient's answers to the questions relating to sensor data may all be negative (e.g., patient 4 has not recently experienced sweating, chills and shivering, headaches, etc.). In such a case, processing circuitry may determine a low risk-level of the patient's health. However, sensor data indicating that the body temperature of patient 4 is 100° F. is above the pre-determined upper range of 99° F. Additionally, at least one of patient's answer to the at least one question relating to sensor data may be positive (e.g., patient 4 has recently experienced sweating, chills and shivering, headaches, etc.). In such a case, processing circuitry may determine that a health problem poses a high risk-level of a condition of the patient's health.

As demonstrated by the above examples, the processing circuitry may determine a risk-level of a patient's health based on temperature. However, it should be understood that processing circuitry may determine a risk-level of a patient's health based on one or more physiological parameters, including, but not limited to, temperature, electrocardiogram (ECG) data, impedance, fluid levels, respiration rate, posture, frequency and duration of activities, heart sounds (e.g., from an accelerometer), perfusion (e.g., from optical sensors), and/or the like. For example, the processing circuitry may determine a high risk-level of a patient's health based on a high blood pressure (e.g., a systolic blood pressure of 150 and a diastolic blood pressure of 120), a high heart rate (e.g., a heart rate of 90 BPM), and so on using the techniques of this disclosure.

In some examples, determining, based on a combination of the sensor data and the at least one answer, a risk-level of the patient's health (38) may include inputting the sensor data and the at least one answer to the at least one question into a decision tree algorithm, which is described in greater detail below.

In some examples, the method may further include monitoring, by the processing circuitry, patient 4, where at least one of a period or a frequency of monitoring is based on the risk level of the patient's health. For example, in response to the risk level of a patient's health being low, processing circuitry may monitor (e.g., collect sensor data) patient 4 once every day and for a period of a minute each monitoring session. Alternatively, in response to the risk level of a patient's health being high, then processing circuitry may collect sensor data about patient 4 more frequently and for longer periods, for example multiple times per day and for a period of multiple minutes during each monitoring session.

In some examples, the method may further include outputting, by the processing circuitry and based on the risk-level of the patient's health, at least one of the at least one question about the patient's health, an indication of the risk level of the patient's health, a notification to maintain status quo, a recommendation to increase monitoring, or an alert to contact a clinician. For example, in response to the risk level of the patient's health being low, processing circuitry may output an indication that the risk level of the patient's health is low and/or a notification to maintain status quo. Alternatively, in response to the risk level of the patient's health being high, processing circuitry may output an indication that the risk level of the patient's health is high, a notification to change the status quo, a recommendation to increase monitoring, and/or an alert to contact a clinician.

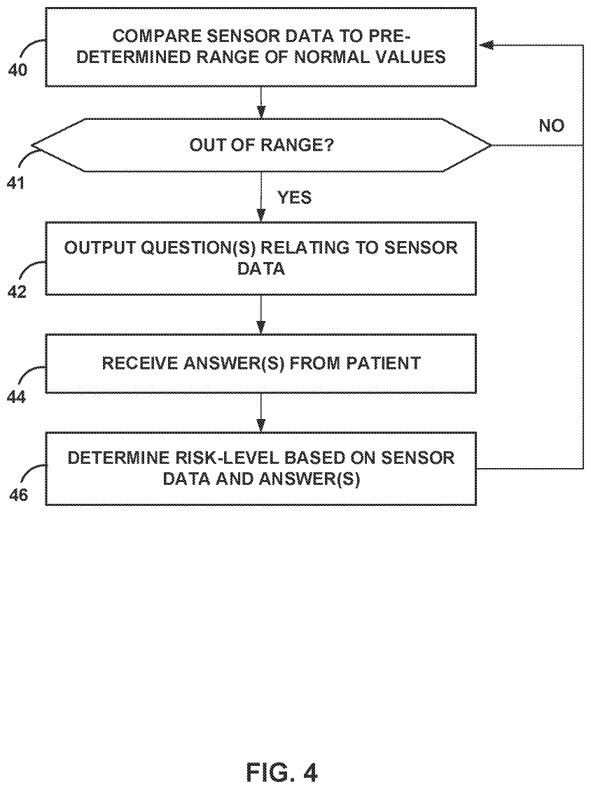

FIG. 4 is a flowchart illustrating an example method of assessing a risk-level of the patient's health. The method may include comparing sensor data, collected by medical device 6 and received by processing circuitry, to a pre-determined range of values (40). Based on the comparison, the processing circuitry determines whether the sensor data is outside of the pre-determined range, e.g., satisfies a threshold that defines the range (41). If the sensor data is within the range (NO of 41), the processing circuitry may continue to compare new sensor data to the range (40). If the sensor data is out of range (YES of 41), the processing circuitry may output at least one question relating to the sensor data (42) and receiving at least one answer from patient 4 (44). The method may further include determining a risk-level of the patient's health based on sensor data and the at least one answer (46).

The pre-determined range of values to which the sensor data is compared may be the range of values for a physiological parameter indicative of normal health. For example, a pre-determined range of values for body temperature may be 97° F. (36.1° C.) to 99° F. (37.2° C.). In such examples, the difference between at least one value of sensor data to the pre-determined range of values may be analyzed. For example, the difference may be analyzed using a decision tree algorithm, which is discussed in further detail below, an artificial intelligence engine, a machine learning model, statistical analysis, a scoring system, a Bayesian Belief Network model, or other analytical technique.

Comparing sensor data to a pre-determined range of values may include determining whether at least one value of sensor data is outside the pre-determined range of values. In such examples, the pre-determined range of values may be the range of values for a physiological parameter indicative of normal health. For example, a pre-determined range of values for body temperature may be 97° F. (36.1° C.) to 99° F. (37.2° C.). In such examples, a sensor data value of 100° F. is outside the pre-determined range of values and may be indicative of abnormal health (e.g., a clinically significant event), such as infection, fever, or another medical condition for which high body temperature is a symptom.

The method may include outputting at least one question relating to the sensor data. Outputting, by the processing circuitry, the at least one question may or may not be in response to at least one sensor data being outside of the pre-determined range of values. For example, the question may be outputted by processing circuitry 20 of peripheral device 2. The questions may be physiological and/or psychological in nature and for the purpose of diagnosing patient 4. The form of the question may be multiple-choice (e.g., ‘T/F’, ‘Y/N’, etc.), free-response (e.g., a sentence, paragraph, etc.), or any other form suitable for soliciting information from patient 4.

The method may include receiving at least one answer from patient 4 to the at least one question outputted (e.g., by peripheral device 2). Patient 4 may provide an input via peripheral device 2 (e.g., using the display of a touch-screen device, a keyboard, an audio recording, etc.). The input may be the selection of a choice from a plurality of choices, a free-response (e.g., a sentence, paragraph, etc.), or any other input suitable for providing information from patient 4.

The method may include determining a risk-level based on sensor data and the at least one answer from patient 4. Risk-level may be in part determined based on the difference between at least one value of sensor data from the pre-determined range of values. For example, a pre-determined range of values for body temperature may be 97° F. (36.1° C.) to 99° F. (37.2° C.). In such examples, a sensor data value of 100° F. is outside the pre-determined range of values. Further, the difference between sensor data value of sensor data value of 100° F. and the upper range of pre-determined values 99° F. is 1° F. The difference of 1° F. may then be evaluated to in part determine the risk-level of patient's health. In such examples, a difference greater than 1° F. may result in a determination of a higher risk-level of patient's health, and a difference less than 1° F. may result in a determination of a lower risk-level of patient's health. In some examples, the processing circuitry may use other data (e.g., ambient temperature measurements, activity data, etc.) in conjunction with the sensor data to determine the risk-level of patient's health. Peripheral device 2 or another device in system 10 may include one or more sensors (e.g., external sensors, such as thermometers) for sensing the other data.

Risk-level may be further determined based on the difference between the at least one answer from the patient to the at least one question and at least one answer indicative of normal health to the same question or questions. For example, processing circuitry 20 may output via peripheral device 2 a question whether patient 4 recently experienced sweating, chills and shivering, headaches, muscle aches, loss of appetite, irritability, dehydration, and/or general weakness to determine if patient 4 is suffering from an infection, fever, or some other medical condition for which a high body temperature is a symptom. In such examples, the answer ‘no’ to whether patient 4 recently experienced any of those symptoms is indicative of normal health. Thus, patient 4 inputting the answer ‘yes’ to at least one question is different from the answer indicative of normal health to the same question or questions, and the at least one difference may be evaluated to determine a risk-level of patient's health. Further, patient 4 inputting the answer ‘yes’ to more of those questions (e.g., patient 4 recently experienced sweating, chills and shivering, headaches, and muscle aches) may be indicative of a higher risk-level of patient's health, whereas patient 4 inputting the answer ‘no’ to more of those questions (e.g., patient 4 has not recently experienced sweating, chills and shivering, headaches, and muscle aches) may be indicative of a lower risk-level of patient's health.

Risk-level of patient's health based on sensor data and at least one answer from patient 4 may be determined using a decision tree algorithm, which is discussed in further detail below, an artificial intelligence engine, a machine learning model, statistical analysis, or other analytical technique. Additionally or alternatively, these analytical techniques may determine the effect of variables unrelated to patient's health but that interfere with sensor data (e.g., a very hot environment or a very cold environment). Risk of level of patient's health may then be determined based on sensor data adjusted to remove or reduce the effect of these variables and the at least one answer from patient 4.

FIG. 5 is a flowchart illustrating an example decision tree algorithm to assess a risk-level of the patient's health. The decision tree algorithm of FIG. 5 may be an example of decision tree algorithm 28 implemented by processing circuitry 20. Decision tree algorithm may include determining, based on sensor data and at least one answer from patient 4 the presence or absence of various factors and/or symptoms relating to a medical condition (e.g., a clinically significant event). The decision tree algorithm may further prescribe at least one question for processing circuitry to output to patient 4 for the purpose of diagnosing patient 4.

For example, processing circuitry 20 may compare sensor data, collected by medical device 6, to a pre-determined range of values (50). If no values of sensor data are indicative of abnormal health (NO of 50), the processing circuitry 20 may determine a relatively low risk-level of patient's health and notify patient 4 of such risk-level, or not provide a notification because the risk-level is low. If at least one value of sensor data is indicative of abnormal health (YES of 50), processing circuitry 20 may ask patient 4 whether patient 4 has recently been in an environment capable of interfering with sensor data (54). If patient 4 answers that patient 4 has recently been in an environment capable of interfering with sensor data (YES of 54), processing circuitry 20 may determine that the risk-level of patient's health is indeterminate due to possibly inaccurate sensor data (56). In such a case, processing circuitry 20 may attempt to determine the risk-level of patient's health by supplementing sensor data with other data (e.g., from an external sensor, such as a thermometer, activity monitor, etc.) to compensate for the inaccuracy of the sensor data. However, it should be understood that in any case processing circuitry 20 may use sensor data in conjunction with other data to determine the risk-level of patient's health.

If instead patient 4 answers that patient 4 has not recently been in an environment capable of interfering with sensor data (NO of 54), processing circuitry 20 may generate an output to ask patient 4 whether patient 4 has recently experienced at least one symptom (e.g., sweating, chills and shivering, etc.) related to a medical condition (e.g., infection) sweating (58). If patient 4 answers that patient 4 has not recently experienced at least one symptom associated with a medical condition (NO of 58), processing circuitry 20 may determine that the risk-level of patient's health is low (or moderate depending on the one or more questions asked and the answers provided by the patient) (60). If patient 4 answers that patient 4 recently experienced at least one symptom related to a medical condition (YES of 58), processing circuitry 20 may increase the risk-level of patient's health from, for example, an initially low risk-level or previously determined risk-level from a prior monitoring session to a higher risk-level (e.g., moderate risk-level of patient's health).

Decision tree algorithm may continue to prescribe questions relating to whether patient experienced at least one symptom associated with a medical condition (58) to patient 4 until an accurate diagnosis can be obtained. For example, after prescribing a question about at least one symptom (e.g., sweating), the decision tree algorithm may continue to prescribe questions to patient about whether patient recently experienced chills and shivering, headaches, muscle aches, etc., increasing risk-level of patient's health (e.g., from a low risk-level to a moderate risk-level) for each additional symptom patient 4 recently experienced (YES of 58), or not changing or decreasing risk-level of patient's health for each additional symptom patient 4 has not recently experienced (NO of 58). Additionally or alternatively, decision tree algorithm may prescribe questions to patient 4 about whether patient 4 is currently experiencing at least one symptom associated with a medical condition and is in physiological and/or psychological distress (62). If patient 4 answers that patient 4 is not currently experiencing at least one symptom associated with a medical condition and is in physiological and/or psychological distress (NO of 62), decision tree algorithm may determine, by processing circuitry 20, a moderate risk-level of patient's health (64). If patient 4 answers that patient 4 is currently experiencing at least one symptom associated with a medical condition and is in physiological and/or psychological distress (YES of 62), decision tree algorithm may determine, by processing circuitry 20, a high risk-level of patient's health (66).

Thus, sensor data, user-reported data (e.g., the at least one answer to the at least one question), and/or other data may be inputted to the decision tree algorithm. According to the decision tree algorithm, various features of the sensor data and various answers may be compared, e.g., in a particular sequence defined by the branching of the decision tree, to various criteria. For example, if patient 4 inputs as an answer that patient 4 is male, the decision tree algorithm may use thresholds for physiological parameters (e.g., heart rate, blood pressure, weight, etc.) associated with males to determine the risk level of the patient's health. Further, the decision tree algorithm may cause processing circuitry 20 to output one or more questions to the patient that are intended for male patients, in this way soliciting more particularized and relevant medical information. Similarly, if patient 4 inputs an answer that patient 4 is female, the decision tree algorithm may determine the risk level of the patient's health by using thresholds for physiological parameters associated with females and/or causing processing circuitry 20 to output one or more questions to the patient that are intended for female patients.

As another example, if the data (e.g., sensor data, user-reported data, etc.) indicates that patient 4 is experiencing a particular medical issue (e.g., heart failure), the decision tree algorithm may cause processing circuitry 20 to output one or more questions (e.g., whether the patient is experiencing chest pain, shortness of breath, etc.) related to that particular medical issue. In some examples, these questions may request that patient 4 measure the particular physiological parameters (e.g., blood pressure, heart rate, etc.) related to the particular medical issue for determining the risk-level of the patient's health. Additionally or alternatively, processing circuitry 20 may cause one or more particular sensors (e.g., the medical device, a blood pressure cuff, a finger pulse oximeter, a glucose meter, etc.) to measure (e.g., continuously or periodically) the particular physiological parameters related to the particular medical issue.

It is to be recognized that while many of the techniques and examples involved diagnosing a patient for infection, the techniques and examples may be used to diagnose a patient for another medical condition, including, but not limited to, stroke, COVID (e.g., COVID-19), cancer, or other conditions. Sensor data for diagnosing a variety of conditions may relate to various physiological parameters, including, but not limited to, temperature, ECG data, oxygen saturation, blood pressure, activity level, caloric expenditure, etc. A range of values indicative of normal health may be pre-determined for each of these physiological parameters, such that techniques in accordance with this disclosure may be applied.

Techniques in accordance with this disclosure may be applied to monitor a patient 4 at risk for stroke (e.g., a transient ischemic attack) based on sensor data (e.g., ECG data, blood pressure, etc.) and user-reported data (e.g., at least one answer to at least one physiological and/or psychological question relating to sensor data). Additionally or alternatively, techniques in accordance with this disclosure may be applied to determine the risk-level of a patient's health for stroke by assessing a change in the CHA₁DS₂-VASc score of patient 4. For example, patient may be prompted to answer at least one question outputted by peripheral device 2 relating to sensor data associated with physiological parameters relating to the CHA₂DS₂-VASc score (e.g., ECG data, blood pressure, etc.). Processing circuitry 20 may then determine a CHA₂DS₂-VASc score based on the sensor data and at least one answer and output, based on the risk-level of the patient's health, at least one of the at least one question about the patient's health, an indication of the risk-level of the patient's health, a notification to maintain status quo, a recommendation to increase monitoring, or an alert to contact a clinician. In some examples, processing circuitry 20 may further determine the risk-level of the patient's health (e.g., the CHA₂DS₂-VASc score) based on patient information from an EMR system.

For example, in response to the risk level of the patient's health being low (e.g., the risk level of the patient experiencing a stroke is low), processing circuitry 20 may output an indication that the risk level of the patient's health is low and/or a notification to maintain status quo. Alternatively, in response to the risk level of the patient's health being high, processing circuitry 20 may output an indication that the risk level of the patient's health is high, a notification to change the status quo, a recommendation to increase monitoring, and/or an alert to contact a clinician. Additionally or alternatively, if processing circuitry 20 determines that the change in the CHA₂DS₂-VASc score is significant (e.g., outside a pre-determined range of values indicative of normal health) processing circuitry 20 may perform a variety of actions, such as notifying patient to contact a health care professional as well as increase frequency of monitoring. In any case, the frequency of monitoring may be tailored (e.g., by the processing circuitry, a clinician, etc.) to the patient's condition and the physiological parameters that medical device 6 is monitoring.

In the case where processing circuitry 20 is not already monitoring patient 4, processing circuitry 20 may begin monitoring patient 4 upon detecting that patient 4 is experiencing a clinically significant event (e.g., an AF episode). In such examples, system 10 may automatically detect the episode based on whether patient 4 belongs to at least one of the classes of AF (e.g., paroxysmal, persistent, long-standing persistent, etc.). Processing circuitry 20 may identify patient 4 as belonging to at least one of these classes based on sensor data and/or patient answers.

Processing circuitry 20 may output a series of questions to determine the CHA₂DS₂-VASc score for patient 4. For example, processing circuitry 20 may use the decision tree algorithm to output a series of questions relating to the age of patient 4 and whether patient has recently experienced symptoms associated with stroke. For example, processing circuitry 20 may, in accordance with the decision tree algorithm, output (e.g., via peripheral device 2) questions relating to whether patient 4 has been diagnosed with heart failure recently and whether patient 4 has been diagnosed with hypertension (HTN). Additionally or alternatively, processing circuitry 20 may prompt (e.g., immediately after outputting questions, at a pre-determined time, upon the triggering of a condition, etc.) patient 4, a caretaker, or another healthcare provider to collect sensor data on physiological parameters of patient 4 (e.g., by using one or more measuring peripheral devices, such as a blood pressure cuff, a finger pulse oximeter, a glucose meter, etc.) In any case, if patient 4 has been diagnosed with HTN, processing circuitry 20, in accordance with the decision tree algorithm, may output a question about whether the patient's blood pressure is outside of normal range (e.g. SBP>130 mmHg) recently. Processing circuitry 20 may further use the decision tree algorithm to output questions about whether patient 4 has been diagnosed with type 2 diabetes recently and/or whether patient 4 has ever had a stroke. Processing circuitry may then determine the CHA₂DS₂-VASc score for patient 4 based on the sensor data and at least one answer to these questions. Additionally, system 10 may update and/or track the CHA₂DS₂-VASc score for patient 4 for a period of time to facilitate determining the health risk of a patient's health, such as the likelihood of the patient experiencing a stroke. For example, medical device 6 may be configured to monitor (e.g., collect sensor data on) the blood pressure of patient 4 for a period of time, and system 10 may use the sensor data from at least a portion of that period of time to determine a hypertension status of patient 4.

Questions relating to the sensor data for each of these physiological parameters and medical conditions involving these physiological parameters may be outputted to patient 4 for purposes of diagnosis. For example, techniques in accordance with this disclosure may be applied to monitor a patient 4 at risk for stroke by, for example, asking patient 4 whether patient recently experienced a particularly exciting event (e.g., riding a roller coaster) that can interfere with sensor data, sudden confusion, trouble speaking, difficulty understanding speech, and other symptoms related to stroke.

Various aspects of the techniques may enable the following examples.

Example 1: A method of monitoring a patient using a system includes receiving sensor data collected by the medical device; evaluating the sensor data; based on the evaluation of the sensor data, outputting for display via the peripheral device at least one question relating to the sensor data collected by the medical device for a patient to answer; receiving at least one answer via the peripheral device; and determining, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.

Example 2: The method of example 1, wherein evaluating the sensor data includes determining that at least one value of the sensor data is outside a pre-determined range of values, and wherein outputting the at least one question includes outputting the at least one question in response to the determination that the at least one value is outside the pre-determined range.

Example 3: The method of any of examples 1 and 2 or 2, wherein the sensor data further includes data collected by at least one of the peripheral device or one or more other measuring peripheral devices.

Example 4: The method of example 3, wherein at least a portion of the sensor data collected by the peripheral device or the one or more other measuring peripheral devices relates to at least one of a location, appearance, or sound of the patient.

Example 5: The method of any of examples 1 to 4, wherein the at least one question relating to the sensor data collected by the medical device for the patient to answer is outputted for display via the peripheral device a pre-determined time period after the processing circuitry evaluates the sensor data.

Example 6: The method of any of examples 1 to 5, wherein the medical device includes at least one of a cardiac monitor or cardiac therapy device.

Example 7: The method of any of examples 1 to 6, wherein the peripheral device includes a smartphone or a tablet.

Example 8: The method of any of examples 1 to 7, wherein the at least one question includes questions regarding at least one of symptoms of an illness or recent activities.

Example 9: The method of any of examples 1 to 8, wherein the sensor data includes at least one of temperature data or electrocardiogram (ECG) data.

Example 10: The method of any of examples 1 to 9, wherein the sensor data includes an average of a series of measurements over time.

Example 11: The method of any of examples 1 to 10, further including confirming, by the processing circuitry, accuracy of the sensor data using the patient's at least one answer to the at least one question.

Example 12: The method of any of examples 1 to 11, wherein determining the risk-level of the patient's health includes inputting, by the processing circuitry, the sensor data and the at least one answer to the at least one question into a decision tree algorithm.

Example 13: The method of any of examples 1 to 12, further including monitoring, by the processing circuitry, the patient, where at least one of a period or a frequency of monitoring is based on the risk level of the patient's health.

Example 14: The method of any of examples 1 to 13, further including outputting for display, by the processing circuitry and based on the risk-level of the patient's health, at least one of the at least one question about the patient's health, an indication of the risk level of the patient's health, a notification to maintain status quo, a recommendation to increase monitoring, or an alert to contact a clinician.

Example 15: The method of any of examples 1 to 14, wherein the sensor data includes temperature data, wherein evaluating the sensor data includes determining whether the temperature data is outside a predetermined temperature range, and wherein determining the risk-level includes determining a risk-level of infection.

Example 16: The method of any of examples 1 to 15, wherein the sensor data includes cardiac electrogram data, wherein evaluating the sensor data includes identifying one or more atrial fibrillation episodes in the sensor data, and wherein determining the risk-level includes determining a risk-level of stroke.

Example 17: The method of any of examples 1 to 16, further including determining, based on the sensor data, a probability of the patient experiencing heart failure, chronic obstructive pulmonary disease, heart failure decompensation, cardiac arrhythmia, or myocardial infarction, and wherein outputting for display via the peripheral device at least one question includes outputting the at least one question based on the probability of the patient experiencing at least one of heart failure, chronic obstructive pulmonary disease, heart failure decompensation, cardiac arrhythmia, or myocardial infarction.

Example 18: The method of example 17, wherein the probability of the patient experiencing at least one of heart failure, chronic obstructive pulmonary disease, heart failure decompensation, cardiac arrhythmia, or myocardial infarction is determined by comparing the sensor data to a set of thresholds.

Example 19: The method of any of examples 1 through 18, wherein at least a portion of the sensor data is related to at least one of an intra-thoracic impedance, an atrial fibrillation, a burden and rate control information, a night heart rate, a heart rate variability, or a patient activity.

Example 20: A system for monitoring a patient includes a medical device configured to collect sensor data related to at least one physiological parameters of the patient; a peripheral device configured to wirelessly communicate with the medical device; and processing circuitry configured to: receive sensor data collected by the medical device; evaluate the sensor data; based on the evaluation of the sensor data, output for display via the peripheral device at least one question relating to the sensor data collected by the medical device for a patient to answer; receive at least one answer via the peripheral device; and determine, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.

Example 21: The system of example 20, wherein the processing circuitry is configured to evaluate the sensor data by determining that at least one value of the sensor data is outside a pre-determined range of values, and wherein the processing circuitry is configured to output the at least one question in response to the determination that the at least one value is outside the pre-determined range.

Example 22: The system of any of examples 20 and 21 or 21, wherein the sensor data further includes data collected by at least one of the peripheral device or one or more other measuring peripheral devices.

Example 23: The system of example 22, wherein at least a portion of the sensor data collected by the peripheral device or the one or more other measuring peripheral devices relates to at least one of a location, appearance, or sound of the patient.

Example 24: The system of examples 20 to 23, wherein the processing circuitry is configured to output at least one question relating to the sensor data for the patient to answer a pre-determined time period after the processing circuitry evaluates the sensor data.

Example 25: The system of any of examples 20 through 24 to 24, wherein the medical device is at least one of a cardiac monitor or cardiac therapy device.

Example 26: The system of any of examples 20 to 25, wherein the peripheral device is a smartphone or a tablet.

Example 27: The system of any of examples 20 to 26, wherein the at least one question includes questions regarding at least one of symptoms of an illness or recent activities.

Example 28: The system of any of examples 20 to 27, wherein the sensor data includes at least one of temperature data or electrocardiogram (ECG) data.

Example 29: The system of any of examples 20 to 28, wherein the sensor data includes an average of a series of measurements over time.

Example 30: The system of any of examples 20 to 29, wherein the processing circuitry is further configured to confirm accuracy of the sensor data using the patient's at least one answer to the at least one question.

Example 31: The system of any of examples 20 to 30, wherein the processing circuitry is further configured to determine the risk-level of the patient's health by inputting the sensor data and the at least one answer to the at least one question into a decision tree algorithm.

Example 32: The system of any of examples 20 to 31, wherein the processing circuitry is further configured to monitor, by the processing circuitry, the patient, where at least one of a period or a frequency of monitoring is based on the risk level of the patient's health.

Example 33: The system of any of examples 20 to 32, wherein the processing circuitry is further configured to output for display, by the processing circuitry and based on the risk level of the patient's health, at least one of the at least one question about the patient's health, an indication of the risk level of the patient's health, a notification to maintain status quo, a recommendation to increase monitoring, or an alert to contact a clinician.

Example 34: The system of any of examples 20 to 33, wherein the sensor data includes temperature data, and wherein the processing circuitry is configured to: determine whether the temperature data is outside a predetermined temperature range; and determine a risk-level of infection.

Example 35: The system of any of examples 20 to 34, wherein the sensor data includes cardiac electrogram data, and wherein the processing circuitry is configured to: identify one or more atrial fibrillation episodes in the sensor data; and determine a risk-level of stroke.

Example 36: A computer-readable medium includes receive sensor data collected by the medical device; evaluate the sensor data; based on the evaluation of the sensor data, output for display via the peripheral device at least one question relating to the sensor data collected by the medical device for a patient to answer; receive at least one answer via the peripheral device; and determine, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

Based upon the above discussion and illustrations, it is recognized that various modifications and changes may be made to the disclosed technology in a manner that does not necessarily require strict adherence to the examples and applications illustrated and described herein. Such modifications do not depart from the true spirit and scope of various aspects of the disclosure, including aspects set forth in the claims.

In at least one example, the functions described may be implemented in hardware, software, firmware, or any combination thereof. If implemented in software, the functions may be stored on or transmitted over as at least one instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by at least one computers or at least one processors to retrieve instructions, code and/or data structures for implementation of the techniques described in this disclosure. A computer program product may include a computer-readable medium.

By way of example, and not limitation, such computer-readable data storage media can include RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Combinations of the above should also be included within the scope of computer-readable media.

Instructions may be executed by at least one processors, such as at least one DSPs, general purpose microprocessors, ASICs, FPGAs, CPLDs, or other equivalent integrated or discrete logic circuitry. Accordingly, the term “processor,” as used herein may refer to any of the foregoing structure or any other structure suitable for implementation of the techniques described herein. Also, the techniques could be fully implemented in at least one circuits or logic elements.

Any of the above-mentioned “processors,” and/or devices incorporating any of the above-mentioned processors or processing circuitry, may, in some instances, be referred to herein as, for example, “computers,” “computer devices,” “computing devices,” “hardware computing devices,” “hardware processors,” “processing units,” “processing circuitry,” etc. Computing devices of the above examples may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In some examples, the computing devices may be controlled by a proprietary operating system. Conventional operating systems control and schedule computer processes for execution, perform memory management, provide file system, networking, I/O services, and provide UI functionality, such as GUI functionality, among other things.

The techniques of this disclosure may be implemented in a wide variety of devices or apparatuses, including an integrated circuit (IC) or a set of ICs (e.g., a chip set). Various components, modules, or units are described in this disclosure to emphasize functional aspects of devices configured to perform the disclosed techniques, but do not necessarily require realization by different hardware units.

Various examples have been described. These and other examples are within the scope of the following claims.

What is claimed is:

1. A system for monitoring a patient, the system comprising:
a medical device configured to collect sensor data related to at least one physiological parameters of the patient;
a peripheral device configured to wirelessly communicate with the medical device; and
processing circuitry configured to:
receive sensor data collected by the medical device;
evaluate the sensor data;
based on the evaluation of the sensor data, output for display via the peripheral device at least one question relating to the sensor data collected by the medical device for a patient to answer;
receive at least one answer via the peripheral device; and
determine, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.

2. The system of claim 1, wherein the processing circuitry is configured to evaluate the sensor data by determining that at least one value of the sensor data is outside a pre-determined range of values, and wherein the processing circuitry is configured to output the at least one question in response to the determination that the at least one value is outside the pre-determined range.

3. The system of claim 1, wherein the sensor data further comprises data collected by at least one of the peripheral device or one or more other measuring peripheral devices.

4. The system of claim 3, wherein at least a portion of the sensor data collected by the peripheral device or the one or more other measuring peripheral devices relates to at least one of a location, appearance, or sound of the patient.

5. The system of claim 1, wherein the processing circuitry is further configured to confirm accuracy of the sensor data using the patient's at least one answer to the at least one question.

6. The system of claim 1, wherein the processing circuitry is further configured to determine the risk-level of the patient's health by inputting the sensor data and the at least one answer to the at least one question into a decision tree algorithm.

7. The system of claim 1, wherein the processing circuitry is further configured to monitor, by the processing circuitry, the patient, where at least one of a period or a frequency of monitoring is based on the risk level of the patient's health.

8. The system of claim 1, wherein the processing circuitry is further configured to output for display, by the processing circuitry and based on the risk level of the patient's health, at least one of the at least one question about the patient's health, an indication of the risk level of the patient's health, a notification to maintain status quo, a recommendation to increase monitoring, or an alert to contact a clinician.

9. The system of claim 1, wherein the sensor data comprises temperature data, and wherein the processing circuitry is configured to:
determine whether the temperature data is outside a predetermined temperature range; and
determine a risk-level of infection.

10. The system of claim 1, wherein the sensor data comprises cardiac electrogram data, and wherein the processing circuitry is configured to:
identify one or more atrial fibrillation episodes in the sensor data; and
determine a risk-level of stroke.

11. A method of monitoring a patient using a system comprising a medical device, a peripheral device configured to wirelessly communicate with the medical device, and processing circuitry, the method comprising, by the processing circuitry:
receiving sensor data collected by the medical device;
evaluating the sensor data;
based on the evaluation of the sensor data, outputting for display via the peripheral device at least one question relating to the sensor data collected by the medical device for a patient to answer;
receiving at least one answer via the peripheral device; and
determining, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.

12. The method of claim 11, wherein evaluating the sensor data comprises determining that at least one value of the sensor data is outside a pre-determined range of values, and wherein outputting the at least one question comprises outputting the at least one question in response to the determination that the at least one value is outside the pre-determined range.

13. The method of claim 11, wherein determining the risk-level of the patient's health comprises inputting, by the processing circuitry, the sensor data and the at least one answer to the at least one question into a decision tree algorithm.

14. The method of claim 11, further comprising outputting for display, by the processing circuitry and based on the risk-level of the patient's health, at least one of the at least one question about the patient's health, an indication of the risk level of the patient's health, a notification to maintain status quo, a recommendation to increase monitoring, or an alert to contact a clinician.

15. The method of claim 11, wherein the sensor data comprises temperature data, wherein evaluating the sensor data comprises determining whether the temperature data is outside a predetermined temperature range, and wherein determining the risk-level comprises determining a risk-level of infection.

16. The method of claim 11, wherein the sensor data comprises cardiac electrogram data, wherein evaluating the sensor data comprises identifying one or more atrial fibrillation episodes in the sensor data, and wherein determining the risk-level comprises determining a risk-level of stroke.

17. The method of claim 11 further comprising determining, based on the sensor data, a probability of the patient experiencing heart failure, chronic obstructive pulmonary disease, heart failure decompensation, cardiac arrhythmia, or myocardial infarction, and wherein outputting for display via the peripheral device at least one question comprises outputting the at least one question based on the probability of the patient experiencing at least one of heart failure, chronic obstructive pulmonary disease, heart failure decompensation, cardiac arrhythmia, or myocardial infarction.

18. The method of claim 17, wherein the probability of the patient experiencing at least one of heart failure, chronic obstructive pulmonary disease, heart failure decompensation, cardiac arrhythmia, or myocardial infarction is determined by comparing the sensor data to a set of thresholds.

19. The method of claim 11, wherein at least a portion of the sensor data is related to at least one of an intra-thoracic impedance, an atrial fibrillation, a burden and rate control information, a night heart rate, a heart rate variability, or a patient activity.

20. A computer-readable medium comprising instructions that, when executed, cause processing circuitry to:
receive sensor data collected by the medical device;
evaluate the sensor data;
based on the evaluation of the sensor data, output for display via the peripheral device at least one question relating to the sensor data collected by the medical device for a patient to answer;
receive at least one answer via the peripheral device; and
determine, based on a combination of the sensor data and the at least one answer, a risk-level of the patient's health associated with at least one of infection, stroke, sepsis, heart failure, chronic obstructive pulmonary disease, heart failure decompensation, cardiac arrhythmia, or myocardial infarction.
